(19) United States
(12) Patent Application Publication
(10) Pub. No.: US 2011/0117080 A1
(43) Pub. Date: May 19, 2011

(54) Anti-REG4 Antibodies

(75) Inventors: Mary J. Janatpour, Castro Valley, CA (US); Kathryn E. Vanderlaag, Sunnyvale, CA (US); Wei Wang, Palo Alto, CA (US)

(73) Assignee: Schering Corporation, Kenilworth, NJ (US)

(21) Appl. No.: 12/945,588

(22) Filed: Nov. 12, 2010

Related U.S. Application Data
(60) Provisional application No. 61/307,769, filed on Feb. 24, 2010.
(60) Provisional application No. 61/261,240, filed on Nov. 13, 2009.

(51) Int. Cl. A61K 39/395; C12P 21/06; G01N 33/53; C12N 5/10; C12N 15/63; C07K 16/18; C07H 21/04

(52) U.S. Cl. 424/130.1; 435/7.1; 435/69.6; 435/325; 435/320.1; 530/387.1; 536/23.53

(57) ABSTRACT

Antibodies to human REG4 are provided, as well as uses thereof, e.g., in treatment of proliferative disorders. Also provided is a method of screening for an antibody that inhibits REG4 bioactivity.

### CROSS-REFERENCE TO RELATED APPLICATIONS

This filing is a U.S. patent application which claims benefit of U.S. Provisional Patent Application No. 61/307,769, filed Feb. 24, 2010, and U.S. Provisional Patent Application No. 61/261,240, filed Nov. 13, 2009, each of which is hereby incorporated by reference in its entirety herein.

### REFERENCE TO SEQUENCE LISTING

The Sequence Listing filed electronically herewith is also hereby incorporated by reference in its entirety (File Name: BP2009_{—}6936US01_SeqListing.txt; Date Created: Nov. 11, 2010; File Size: 28.9 KB.)

### FIELD OF THE INVENTION

The present invention relates generally to antibodies specific for Regenerating islet-derived Gene Type IV (REG4) protein and uses thereof. More specifically, the invention relates to humanized antibodies that recognize human REG4 and modulate its activity, particularly in proliferative disorders, including cancer.

### BACKGROUND OF THE INVENTION

The human Regenerating islet-derived Gene (“REG”) family of ligands consists of four secreted and structurally unique proteins that share sequence similarity with the carbohydrate-binding domain of C-type lectins. The initial cDNA in this gene family was named REG for its role in islet of Langerhans regeneration following partial pancreatectomy (now known as REG Iα). Additional members of the human REG gene family are regenerating gene homologue (REG Iβ) and pancreatitis-associated protein (Reg III). All are constitutively expressed in the normal proximal gastrointestinal tract. While the function of this gene family is poorly understood, recent data has suggested that REG family members may function as tissue mitogens. REG Iα is mitogenic for gastric mucosal cells (Fukui et al. (1998) Gastroenterol. 115:1483-1493), and pancreatic ductal and beta cells (Zenilman et al. (1996) Gastroenterology 110:1208-1214; Zenilman et al. (1998) Pancreas 17:256-261; Watanabe (1994) Proc. Nat'l. Acad. Sci. 91:3589-1392). The serum concentration of REG Iα is significantly increased in many gastrointestinal malignancies, including gastric and pancreatic adenocarcinoma (Satomura et al. (1995) J Gastroenterol. 30:643-650). For patients with early stage colonic adenocarcinoma undergoing surgical resection, REG Iα mRNA expression alone or co-expression of REG Iα and REG III mRNA by the carcinoma had an adverse effect on disease free survival that was independent of tumor stage or site (Macadam et al. (2000) British J Cancer 83:188-95).

A novel member of this gene family, REG IV or REG4, which has significant constitutive expression in the distal gastrointestinal tract was identified by Hartupee et al. (2001) Biochim. Biophys. Acta 1518:287-293. Through molecular modeling, it was shown that the REG4 protein showed maintenance of the conserved contact surface residues that cluster on a single face of the 3-dimensional molecule present in all other members of the REG gene family. This suggests that REG proteins may share similar physiologic actions. Reg IV is of considerable interest because of its possible role, along with other members of the Reg gene family in the pathogenesis of colorectal adenocarcinoma. REG IV is overexpressed by a majority of colorectal adenocarcimonas. By differential display, REG IV was among several genes with increased mRNA expression in several colon cancer cell lines selected for increased in vitro resistance to a cancer chemotherapeutic agent, 5-FU (Violette et al. (2003) Int J. Cancer 103:185-193).

Key to the initiation and progression of cancer is the sustained activation of tyrosine kinase signaling pathways, which relay signals for growth, survival, migration and differentiation. Receptor tyrosine kinases (RTKs) constitute a family of receptors that include epidermal growth factor receptor (EGFR), insulin receptor (IR), insulin-like growth factor receptor (IGF1R) and exist as monomers in an inactive state. Either through mutation or upon extracellular ligand binding, they form homo or heterodimers, which leads to autophosphorylation of the intracellular domain and ultimately results in activation of downstream signaling pathways, including Ras/mitogen activated protein kinase (MAPK) and phosphatidylinositol-3-kinase (PI3-K) pathways. Given the critical cell functions regulated by RTKs, both small molecule and monoclonal antibodies directed against this receptor family have been generated and are currently used to treat cancer patients {ref}. However, like many monotherapies, targeting a single RTK pathway is often not sufficient for complete tumor regression. Consequently, a high percentage of cancers acquire resistance to these treatments, which necessitates identifying other key players in cancer biology.

It has been reported that REG4 activates eipdermal growth factor receptor (EGFR), protein kinase B/Akt and activator protein-1 to accelerate colorectal cancer cell survival by increasing Bcl-2, Bcl-XL and survivin (Bishnupuri et al. (2006) Gastroenterol. 130:137-149). The anti-apoptotic property of Reg IV is associated with colorectal cancer development and drug resistance in gastric cancer (see, e.g., Bishnupuri et al. (2006) Cancer Biol. Ther 5:1714-1720; and Mitani et al. (2007) Oncogene 26:4383-4393) and its expression is expected to be a marker for highly malignant potential (see, e.g., Gu et al. (2005) Clin. Cancer Res. 11:2237-2243; Heiskala et al. (2006) Virchows Arch. 448:295-300: and Pankova-Kholmyansky and Arber (2007). Cancer Biol. Ther. 6:23-124). Recently, REG4 protein was found in the peritoneal lavage fluid of gastric cancer patients at the time of surgery. REG4 protein was detected in macroscopical and cytological peritoneal metastasis, suggesting that REG4 might be a sensitive marker for metastasis of gastric cancer (see, e.g., Kuniyasu et al. (2009) Cell Prolif. 42:110-121). Recently it has been suggested that REG4 is an independent prognostic factor of relapse in prostate cancer (see, e.g, Ohara et al (2008) Cancer Sci. 99:1570-1577).

Many current drugs on the market or in clinical trials are designed to target one RTK, however, human cancer cells simultaneously activate numerous growth factor receptors. In addition, since acquired resistance to EGFR inhibitors can involve upregulation of IGF1R and alternative growth factor pathways, hitting multiple RTKS simultaneously may delay drug resistance. Due to the importance of IR in glucose homeostasis, an ideal anti-IGF strategy would target both IGFIR and IR only in tumor cells. Given the restricted expression profile of Reg IV in normal tissues, antagonizing Reg4 activity might represent a way to target IR only in tumors.

To date, information about REG4 activity has been limited to expression in various tumors and cancers. Little has been described about the biology of REG4 and its modulation of downstream molecules in cancer. The data below suggests that REG4 plays a significant functional role in cancer, supported by evidence of modulation of downstream cancer relevant pathways. Thus, the need exists for improved methods and compositions for the treatment of proliferative disorders by modulating REG4 activity. Preferably, such compositions would have a high affinity for the target molecule, and would be able to modulate REG4 activity at relatively low doses. Preferably, such methods and compositions would be highly specific for REG4, and not interfere with the activity of other receptors. Preferably, such methods and compositions would employ antagonists suitable for modification for the delivery of cytotoxic payloads to target cells, but also suitable for non-cytotoxic uses. Preferably, such methods and compositions would employ antibodies modified to limit their antigenicity when administered to a subject in need thereof.

### SUMMARY OF THE INVENTION

The present invention meets these needs in the art and more by providing antagonists of REG4, e.g. REG4 antibodies.

In one aspect the invention provides binding compounds, such as an antibody or fragment thereof, including humanized or chimeric recombinant antibodies, that binds human REG4, comprising an antibody light chain variable domain, or antigen binding fragment thereof, having at least one or more CDRs selected from the group consisting of SEQ ID NOs: 41-64 and a heavy chain variable domain, having at least one or more CDRs selected from the group consisting of SEQ ID NOs: 17-40.

In other embodiments the binding compound of the present invention comprises a light chain variable domain and a heavy chain variable domain, or the antigen binding fragments thereof, described in the preceding paragraph.

In some embodiments, the binding compound comprises a framework region, wherein the amino acid sequence of the framework region is all or substantially all of a human immunoglobulin amino acid sequence.

In some embodiments the light chain variable domain comprises a sequence selected from the group consisting of SEQ ID NOs: 9-16 or a variant thereof. In some embodiments the heavy chain variable domain comprises a sequence selected from the group consisting of SEQ ID NOs: 1-8. In yet a further embodiment, the binding compound comprises a light chain variable domain and a heavy chain variable domain, or the antigen binding fragments thereof, described in this paragraph.

In one embodiment, the invention relates to antibodies or fragments thereof that are able to block the binding of a binding compound of the present invention to human REG4 in a cross-blocking assay. In various embodiments the antibody is able to block binding of human REG4 to an antibody comprising the CDR sequences of antibodies 1C11, 3C2.3D10, 4C5.3B10, 9F3.3A4, 12E1.3C11, 13E1.1B11, 40F6.3F6, 70A9.3C2, and 86C1.2B7, as disclosed herein.

In some embodiments, the binding compound of the present invention comprises a humanized antibody comprising the CDRs, or variants thereof, selected from the CDRs of the antibodies disclosed herein, in combination with human germline light chain and heavy chain variable domain framework sequences in place of the rodent frameworks of the parental antibodies.

In some embodiments, the binding compound of the present invention further comprises a heavy chain constant region, wherein the heavy chain constant region comprises a γ1, γ2, γ3, or γ4 human heavy chain constant region or a variant thereof. In various embodiments the light chain constant region comprises a lambda or a kappa human light chain constant region.

In various embodiments the binding compounds of the present invention are polyclonal, monoclonal, chimeric, humanized or fully human antibodies or fragments thereof. The present invention also contemplates that the antigen binding fragment is an antibody fragment selected from the group consisting of Fab, Fab′, Fab′-SH, Fv, scFv, F(ab′)₂, and a diabody.

In some embodiments, the antibody specific for REG4 is the humanized or chimeric antibody. The present invention encompasses an isolated nucleic acid encoding the polypeptide sequence of an antibody embodiment of the binding compound of the present invention. The nucleic acid can be in an expression vector operably linked to control sequences recognized by a host cell transfected with the vector. Also encompassed is a host cell comprising the vector, and a method of producing a polypeptide comprising culturing the host cell under conditions wherein the nucleic acid sequence is expressed, thereby producing the polypeptide, and recovering the polypeptide from the host cell or medium.

The present invention encompasses a method of inhibiting a proliferative response in a human subject comprising administering to a subject in need thereof an antibody (or a antigen binding fragment thereof) specific for REG4 in an amount effective to inhibit REG4 activity. In a further embodiment, the antibody can inhibit angiogenesis associated with a cancer or tumor cell in a human subject.

The present invention encompasses a pharmaceutical composition comprising an REG4 antibody or fragment thereof and a pharmaceutically acceptable carrier.

The present invention provides a method of screening for an inhibitor of REG4 comprising: a) contacting a cell with a REG4 antagonist; b) measuring at least one phosphorylation event in the cell; and c) selecting the REG4 inhibitor that decreases at least one phosphorylation event in the cell. In certain embodiments, the REG4 antagonist is a REG4 antibody or fragment thereof.

### DETAILED DESCRIPTION

As used herein, including the appended claims, the singular forms of words such as “a,” “an,” and “the,” include their corresponding plural references unless the context clearly dictates otherwise. Table 10 below provides a listing of sequence identifiers used in this application. All references cited herein are incorporated by reference to the same extent as if each individual publication, database entry (e.g. Genbank sequences or GeneID entries), patent application, or patent, was specifically and individually indicated to be incorporated by reference. Citation of the references herein is not intended as an admission that any of the foregoing is pertinent prior art, nor does it constitute any admission as to the contents or date of these publications or documents.

### DEFINITIONS

The terms “REG4”, “Regenerating islet-derived Gene Type IV”, and “REG IV” are well known in the art. The human REG4 nucleotide and polypeptide sequences are disclosed in e.g., U.S. Pat. No. 5,861,494, U.S. Pat. No. 6,080,722, and U.S. Pat. No. 7,132,509. GenBank® deposits of the human REG4 amino (NP_{—}114433) and nucleic acid (NM_{—}032044) sequences are also available.

“Proliferative activity” encompasses an activity that promotes, that is necessary for, or that is specifically associated with, e.g., normal cell division, as well as cancer, tumors, dysplasia, cell transformation, metastasis, and angiogenesis.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts REG4, e.g., in the fluid phase or colloidal phase, but also situations where the agonist or antagonist does not contact the cell or the receptor.

As used herein, the term “antibody” refers to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, the terms “REG4 binding fragment,” “binding fragment thereof” or “antigen binding fragment thereof” encompass a fragment or a derivative of an antibody that still substantially retains its biological activity of inducing REG4 signaling referred to herein as “REG4 inducing activity.” The term “antibody fragment” or REG4 binding fragment refers to a portion of a full length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its REG4 antagonist activity. Preferably, a binding fragment or derivative retains at least 25%, 50%, 60%, 70%, 80%, 90%, 95%, 99% or 100% (or more) of its REG4 antagonist activity, although any binding fragment with sufficient affinity to exert the desired biological effect will be useful. It is also intended that a REG4 binding fragment can include variants having conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The antibodies of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in the a targeted cell.

The antibodies of the present invention also include antibodies conjugated to cytotoxic payloads, such as cytotoxic agents or radionuclides. Such antibody conjugates may be used in immunotherapy in conjunction with anti-REG4 treatment, to selectively target and kill cells expressing certain antigens on their surface. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. e.g., U.S. Patent Application Publication No. 2006/0014225.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

As used herein, the term “hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196: 901-917). As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding composition with a target where the association results in reduction in the normal Brownian motion of the binding composition, in cases where the binding composition can be dissolved or suspended in solution. “Binding composition” refers to a molecule, e.g. a binding compound, in combination with a stabilizer, excipient, salt, buffer, solvent, or additive, capable of binding to a target.

“Conservatively modified variants” or “conservative substitution” refers to substitutions of amino acids are known to those of skill in this art and may often be made even in essential regions of the polypeptide without altering the biological activity of the resulting molecule. Such exemplary substitutions are preferably made in accordance with those set forth in Table 1 as follows:

TABLE 1
Exemplary Conservative Amino Acid Substitutions

| Original | Conservative |
|---|---|
| residue | substitution |
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Those of skill in this art recognize that, in general, single amino acid substitutions in non-essential regions of a polypeptide may not substantially alter biological activity. See, e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Effective amount” encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health of the patient, the method route and dose of administration and the severity of side affects. See, e.g., U.S. Pat. No. 5,888,530. An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

The terms “cancer”, “tumor”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes, et al. (2006) Br. J. Cancer 94:1057-1065), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer and cancers of mucinous origins, such as, mucinous ovarian cancer, cholangiocarcinoma (liver) and renal papillary carcinoma.

As cancerous cells grow and multiply, they form a mass of cancerous tissue, that is a tumor, which invades and destroys normal adjacent tissues. Malignant tumors are cancer. Malignant tumors usually can be removed, but they may grow back. Cells from malignant tumors can invade and damage nearby tissues and organs. Also, cancer cells can break away from a malignant tumor and enter the bloodstream or lymphatic system, which is the way cancer cells spread from the primary tumor (i.e., the original cancer) to form new tumors in other organs. The spread of cancer in the body is called metastasis (What You Need to Know About Cancer—an Overview, NIH Publication No. 00-1566; posted Sep. 26, 2000, updated Sep. 16, 2002 (2002)).

As used herein, the term “solid tumor” refers to an abnormal growth or mass of tissue that usually does not contain cysts or liquid areas. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

As used herein, the term “primary cancer” refers to the original tumor or the first tumor. Cancer may begin in any organ or tissue of the body. It is usually named for the part of the body or the type of cell in which it originates (Metastatic Cancer: Questions and Answers, Cancer Facts 6.20, National Cancer Institute, reviewed Sep. 1, 2004 (2004)).

As used herein, the term “carcinoma in situ” refers to cancerous cells that are still contained within the tissue where they started to grow, and have not yet become invasive or spread to other parts of the body.

As used herein, the term “carcinomas” refers to cancers of epithelial cells, which are cells that cover the surface of the body, produce hormones, and make up glands. Examples of carcinomas are cancers of the skin, lung, colon, stomach, breast, prostate and thyroid gland.

As used herein, the term “isolated nucleic acid molecule” refers to a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences involved in the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, “polymerase chain reaction” or “PCR” refers to a procedure or technique in which minute amounts of a specific piece of nucleic acid, RNA and/or DNA, are amplified as described in, e.g., U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

As used herein, the term “germline sequence” refers to a sequence of unrearranged immunoglobulin DNA sequences, including rodent (e.g. mouse) and human germline sequences. Any suitable source of unrearranged immunoglobulin DNA may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

To examine the extent of enhancement of REG4 activity, for example, samples or assays comprising a given, e.g., protein, gene, cell, or organism, are treated with a potential activating or inhibiting agent and are compared to control samples without the agent. Control samples, i.e., not treated with agent, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression or modification of a gene or protein relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. As used herein, an antibody is said to bind specifically to a polypeptide comprising a given sequence (in this case REG4) if it binds to polypeptides comprising the sequence of REG4 but does not bind to proteins lacking the sequence of REG4. For example, an antibody that specifically binds to a polypeptide comprising REG4 may bind to a FLAG®-tagged form of REG4 but will not bind to other FLAG®-tagged proteins.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

### General

The present invention provides engineered anti-REG4 antibodies and uses thereof to treat proliferative disorders, in particular cancers and tumors. The expression of REG4, as noted above, is upregulated in the progression of proliferative diseases.

Data from the present invention suggests that neutralizing REG4 activity will be beneficial for cancer patients whose cancers express the REG4 protein, including, but not limited to, GI tract cancers (including colorectal, pancreatic, esophageal, and gastric), ovarian, and prostate cancers.

Using siRNA mediated knockdown to model the effects of neutralizing REG4 activity, it was confirmed that expansion of cell numbers (colorectal and prostate cancer lines) on plastic was inhibited. In addition to the impact on proliferation, there was a demonstrated inhibitory effect of REG4 knockdown in cell cycle. Conversely, an enhancement of the growth of a cell line that does not make REG4 endogenously (HCT116), resulted when exogenous recombinant REG4 was administered.

The proliferation findings were extended by demonstrating that REG4 protein knockdown inhibited anchorage-independent growth, as measured by colony growth from single cells in soft-agar, as well as spheroid growth in suspension. Soft agar growth and spheroid growth assays are more relevant to cancer biology than growth on plastic. It was further demonstrated that REG4 knockdown induces apoptosis in these cell lines suggesting that neutralizing REG4 will not only be cytostatic for cancer cells, but will cause tumor regression.

While the primary focus of these studies was on the growth and survival of the tumor cell, studies to explore other cancer relevant biologies were also undertaken. Consistent with a potential role in angiogenesis, rhREG4 potentiated tubule formation in HUVECs in vitro, suggesting that REG4 is not only important for the growth and survival of tumor cells directly, but can also aid the tumor in recruiting a blood supply.

In addition to studying the phenotypic consequences of neutralizing REG4 in cancer cells, the mechanism of action by which REG4 might be acting was also studied. It had been previously shown that REG4 treatment or over-expression leads to increased phosphorylation of EGFR (see, e.g., Bishhupuri (2006) supra). The data of the present invention indicates the opposite effect, that neutralization of REG4 in PC3 cells, leads to decreased pEGFR, it was also observed that this effect was not universal, but seemingly cell line specific. For example, in a different cancer line (KM12), it was observed that REG4 knockdown led to decreased levels of two other receptor tyrosine kinases (RTKs), phosphor-insulin receptor and phosphor-IGF1R, but not pEGFR. Phosphor-insulin receptor and phosphor-IGF1R in PC3 cells were downregulated. Data of the present invention indicated that recombinant REG4 (mutated for stability) can induce phosphorylation events of several RTKs, including IGF1R and IR, as well as VEGFR and HGF receptor. Collectively, these observations indicate that REG4 expression in cancers may lead to both autocrine and paracrine transactivation of receptor tyrosine kinases that are cancer drivers. The fact that that REG4 may drive phosphorylation events of multiple RTKs is important in that it suggests that targeting, e.g., inhibiting, one protein could impact multiple cancer-relevant pathways. This is distinct from the information known publicly, which has only demonstrated an impact on EGFR, a pathway for which there are already multiple therapeutics in the clinic.

The impact of REG4 on phosphor-insulin receptor and phosphor-IGF1R is important because both of these receptors are cancer drivers. However, targeting the insulin receptor universally could have toxicity issues, due to the widespread expression of this receptor. In contrast, by targeting these RTKs indirectly through REG4, only those cells dependent on REG4 will be affected, thus drastically impacting the potential toxicity profile in a positive manner.

Among the challenges encountered in trying to design IGF1R-specific inhibitors, i.e., inhibitors that do not bind to the highly homologous insulin receptor, is that there is potential for decreased efficacy. The present data suggest that targeting REG4 will be superior to targeting the individual RTKs.

Consistent with additional previous observations, it was shown that REG4 modulation (with knockdown of endogenous REG4 protein and treatment with recombinant REG4 protein) resulted in modulation of pAkt and Bcl-2. In contrast to published information, it was demonstrated that Bcl-2 levels were not modulated at the transcriptional level, but rather at the protein level. The present data also shows that other proteins, not previously associated with REG4 modulation, appear to be regulated by REG4. These include c-Fos, E2F-1, cyclin D1, pERK1, pERK2, GSK3beta, p-p70 S6 kinase, RSK1, MSK2 & HSP27. Furthermore, the present data suggest that ERK inhibition results in decreased REG4 levels. Together, these results suggest a hypothesis that REG4 is up-regulated in cancer to allow survival, enhance growth and perhaps stimulate motility of cancer cells, when these cells would otherwise respond to signals to die.

Lastly, it was observed that REG4 activity modulated HSP27, suggesting an additional mechanism by which REG4 can mediate chemo-resistance analogous to the modulation of DPYD previously observed (see, e.g., Mitani, et al (2007) supra). Therefore, a REG4 antagonistic therapeutic combined with either current or future chemotherapeutics, biologics or immunotherapeutics, should result in increased tumor regression.

Antibodies of the present invention demonstrated the ability to inhibit exogenous REG4 induced proliferation f HCT116 cells. Additionally these antibodies can inhibit growth of the PC3 cancer cell line that endogenously expresses REG4. Combining these antibodies with small molecule inhibitors of pathways downstream of REG4 increased the efficacy of either the antibodies or the small molecule inhibitors alone.

### Generation of REG4 Specific Antibodies

Any suitable method for generating monoclonal antibodies may be used. For example, a recipient may be immunized with REG4 or a fragment thereof. Any suitable method of immunization can be used. Such methods can include adjuvants, other immunostimulants, repeated booster immunizations, and the use of one or more immunization routes. Any suitable source of REG4 can be used as the immunogen for the generation of the non-human antibody of the compositions and methods disclosed herein. Such forms include, but are not limited whole protein, peptide(s), and epitopes generated through recombinant, synthetic, chemical or enzymatic degradation means known in the art.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

Any suitable method can be used to elicit an antibody with the desired biologic properties to inhibit REG4 activity. It is desirable to prepare monoclonal antibodies (mAbs) from various mammalian hosts, such as mice, rats, other rodents, humans, other primates, etc. Description of techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a antigen binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. supra; and Ward et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance that provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. See also Abgenix and Medarex technologies.

Antibodies or binding compositions against predetermined fragments of REG4 can be raised by immunization of animals with conjugates of the polypeptide, fragments, peptides, or epitopes with carrier proteins. Monoclonal antibodies are prepared from cells secreting the desired antibody. These antibodies can be screened for binding to normal or defective REG4. These monoclonal antibodies will usually bind with at least a K_{d} of about 1 μM, more usually at least about 300 nM, 30 nM, 10 nM, 3 nM, 1 nM, 300 pM, 100 pM, 30 pM or better, usually determined by ELISA. Suitable non-human antibodies may also be identified using the biologic assays described in Examples 5 and 6, below.

### Humanization of REG4 Specific Antibodies

Any suitable non-human antibody can be used as a source for the hypervariable region. Sources for non-human antibodies include, but are not limited to, murine (e.g. Mus musculus), rat (e.g. Rattus norvegicus), Lagomorphs (including rabbits), bovine, and primates. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance of the desired biological activity. For further details, see Jones et al. (1986) Nature 321:522-525; Reichmann et al. (1988) Nature 332:323-329; and Presta (1992) Curr. Op. Struct. Biol. 2:593-596.

Methods for recombinantly engineering antibodies have been described, e.g., by Boss et al. (U.S. Pat. No. 4,816,397), Cabilly et al. (U.S. Pat. No. 4,816,567), Law et al. (European Patent Application Publication No. 438310) and Winter (European Patent Application Publication No. 239400).

Amino acid sequence variants of humanized anti-REG4 antibody are prepared by introducing appropriate nucleotide changes into the humanized anti-REG4 antibody DNA, or by peptide synthesis. Such variants include, for example, deletions from, and/or insertions into, and/or substitutions of, residues within the amino acid sequences shown for the humanized anti-REG4 antibody. Any combination of deletion, insertion, and substitution is made to arrive at the final construct, provided that the final construct possesses the desired characteristics. The amino acid changes also may alter post-translational processes of the humanized anti-REG4 antibody, such as changing the number or position of glycosylation sites.

A useful method for identification of certain residues or regions of the humanized anti-REG4 antibody polypeptide that are preferred locations for mutagenesis is called “alanine scanning mutagenesis,” as described by Cunningham and Wells (1989) Science 244: 1081-1085. Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with REG4 antigen. The amino acid residues demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed humanized anti-REG4 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include humanized anti-REG4 antibody with an N-terminal methionyl residue or the antibody fused to an epitope tag. Other insertional variants of the humanized anti-REG4 antibody molecule include the fusion to the N- or C-terminus of humanized anti-REG4 antibody of an enzyme or a polypeptide that increases the serum half-life of the antibody.

Another type of variant is an amino acid substitution variant. These variants have at least one amino acid residue in the humanized anti-REG4 antibody molecule removed and a different residue inserted in its place. The sites of greatest interest for substitutional mutagenesis include the hypervariable loops, but FR alterations are also contemplated.

Another type of amino acid variant of the antibody alters the original glycosylation pattern of the antibody. By altering is meant deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Yet another type of amino acid variant is the substitution of residues to provide for greater chemical stability of the final humanized antibody. For example, an asparagine (N) residue may be changed to reduce the potential for formation of isoaspartate at any NG sequences within a rodent CDR. A similar problem may occur at a DG sequence. Reissner and Aswad (2003) Cell. Mol. Life Sci. 60:1281. Isoaspartate formation may debilitate or completely abrogate binding of an antibody to its target antigen. Presta (2005) J. Allergy Clin. Immunol. 116:731 at 734. In one embodiment, the asparagine is changed to glutamine (Q). In addition, methionine residues in rodent CDRs may be changed to reduce the possibility that the methionine sulfur would oxidize, which could reduce antigen binding affinity and also contribute to molecular heterogeneity in the final antibody preparation. Id. In one embodiment, the methionine is changed to alanine (A). Antibodies with such substitutions are subsequently screened to ensure that the substitutions do not decrease REG4 binding affinity to unacceptable levels.

Nucleic acid molecules encoding amino acid sequence variants of humanized REG4 specific antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of humanized anti-REG4 antibody.

Ordinarily, amino acid sequence variants of the humanized anti-REG4 antibody will have an amino acid sequence having at least 75% amino acid sequence identity with the original humanized antibody amino acid sequences of either the heavy or the light chain more preferably at least 80%, more preferably at least 85%, more preferably at least 90%, and most preferably at least 95%, 98% or 99%. Identity or homology with respect to this sequence is defined herein as the percentage of amino acid residues in the candidate sequence that are identical with the humanized anti-REG4 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

The humanized antibody can be selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Variants of the IgG isotypes are also contemplated. The humanized antibody may comprise sequences from more than one class or isotype. Optimization of the necessary constant domain sequences to generate the desired biologic activity is readily achieved by screening the antibodies in the biological assays described in the Examples.

Likewise, either class of light chain can be used in the compositions and methods herein. Specifically, kappa, lambda, or variants thereof are useful in the present compositions and methods.

Any suitable portion of the CDR sequences from the non-human antibody can be used. The CDR sequences can be mutagenized by substitution, insertion or deletion of at least one residue such that the CDR sequence is distinct from the human and non-human antibody sequence employed. It is contemplated that such mutations would be minimal. Typically, at least 75% of the humanized antibody residues will correspond to those of the non-human CDR residues, more often 90%, and most preferably greater than 95%.

Any suitable portion of the FR sequences from the human antibody can be used. The FR sequences can be mutagenized by substitution, insertion or deletion of at least one residue such that the FR sequence is distinct from the human and non-human antibody sequence employed. It is contemplated that such mutations would be minimal. Typically, at least 75% of the humanized antibody residues will correspond to those of the human FR residues, more often 90%, and most preferably greater than 95%, 98% or 99%.

CDR and FR residues are determined according to the standard sequence definition of Kabat. Kabat et al. (1987) Sequences of Proteins of Immunological Interest, National Institutes of Health, Bethesda Md. SEQ ID NOs: 1-8 show the heavy chain variable domain sequences of various rodent anti-human REG4 antibodies, and SEQ ID NOs: 9-16 depict the light chain variable domain sequences.

TABLE 2
Heavy Chain Sequences and Domains

| | SEQ | | HEAVY CHAIN CDR | | |
|---|---|---|---|---|---|
| ANTIBODY | ID | | RESIDUES | | |
| CLONE | NO: | V_{H} RESIDUES | CDR-H1 | CDR-H2 | CDR-H3 |
| 3C2.3D10 | 1 | 1-141 | 45-54 | 69-85 | 118-130 |
| 4C5.3B10 | 2 | 1-141 | 46-54 | 69-85 | 118-130 |
| 9F3.3A4 | 3 | 1-141 | 45-54 | 69-85 | 118-129 |
| 12E1.3C11 | 4 | 1-134 | 44-53 | 68-83 | 116-123 |
| 13E1.1B11 | 5 | 1-138 | 45-54 | 69-85 | 118-126 |
| 40F6.3F6 | 6 | 1-141 | 45-54 | 69-85 | 119-130 |
| 70A9.3C2 | 7 | 1-135 | 45-54 | 69-85 | 118-124 |
| 86C1.2B7 | 8 | 1-135 | 45-54 | 69-85 | 118-124 |

TABLE 3
Light Chain sequences and Domains

| ANTIBODY | SEQ ID | V_{L} | LIGHT CHAIN CDR RESIDUES | | |
|---|---|---|---|---|---|
| CLONE | NO: | RESIDUES | CDR-L1 | CDR-L2 | CDR-L3 |
| 3C2.3D10 | 9 | 1-132 | 43-58 | 74-80 | 113-121 |
| 4C5.3B10 | 10 | 1-132 | 43-58 | 74-80 | 113-121 |
| 9F3.3A4 | 11 | 1-132 | 43-58 | 74-80 | 113-121 |
| 12E1.3C11 | 12 | 1-128 | 44-54 | 70-76 | 109-117 |
| 13E1.1B11 | 13 | 1-134 | 44-60 | 76-82 | 115-123 |
| 40F6.3F6 | 14 | 1-132 | 43-58 | 74-80 | 113-121 |
| 70A9.3C2 | 15 | 1-128 | 44-54 | 70-76 | 109-117 |
| 86C1.2B7 | 16 | 1-132 | 43-58 | 74-80 | 113-121 |

In one embodiment, CDRs include variants of any single sequence CDR disclosed herein (SEQ ID NOs: 17-64), in which the variant comprises 1, 2, 3, 4, 5, 6, 7, 8, 9, or more conservative amino acid substitutions relative to the disclosed sequence, as determined using the data of Table 1.

Also contemplated are chimeric antibodies. As noted above, typical chimeric antibodies comprise a portion of the heavy and/or light chain identical with, or homologous to, corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. See U.S. Pat. No. 4,816,567; and Morrison et al. (1984) Proc. Natl. Acad. Sci. USA 81: 6851-6855.

Bispecific antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

In yet other embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

The parental and engineered forms of the antibodies of the present invention may also be conjugated to a chemical moiety. The chemical moiety may be, inter alia, a polymer, a radionuclide or a cytotoxic factor. Preferably the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee et al., (1999) (Bioconj. Chem. 10:973-981) discloses PEG conjugated single-chain antibodies. Wen et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments or the REG4 soluble proteins or fragments thereof of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments or the REG4 soluble proteins or fragments thereof of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the invention to the various moieties may be employed, including those methods described by Hunter et al., (1962) Nature 144:945; David et al., (1974) Biochemistry 13:1014; Pain et al., (1981) J. Immunol. Meth. 40:219; and Nygren, J., (1982) Histochem. and Cytochem. 30:407. Methods for conjugating antibodies and proteins are conventional and very well known in the art.

### Antibody Production

In one embodiment, for recombinant production of the antibodies of the present invention, the nucleic acids encoding the two chains are isolated and inserted into one or more replicable vectors for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. In one embodiment, both the light and heavy chains of a humanized anti-REG4 antibody of the present invention are expressed from the same vector, e.g. a plasmid or an adenoviral vector.

Antibodies of the present invention may be produced by any method known in the art. In one embodiment, antibodies are expressed in mammalian or insect cells in culture, such as chinese hamster ovary (CHO) cells, human embryonic kidney (HEK) 293 cells, mouse myeloma NSO cells, baby hamster kidney (BHK) cells, Spodoptera frugiperda ovarian (Sf9) cells. In one embodiment, antibodies secreted from CHO cells are recovered and purified by standard chromatographic methods, such as protein A, cation exchange, anion exchange, hydrophobic interaction, and hydroxyapatite chromatography. Resulting antibodies are concentrated and stored in 20 mM sodium acetate, pH 5.5.

In another embodiment, the antibodies of the present invention are produced in yeast according to the methods described in WO2005/040395. Briefly, vectors encoding the individual light or heavy chains of an antibody of interest are introduced into different yeast haploid cells, e.g. different mating types of the yeast Pichia pastoris, which yeast haploid cells are optionally complementary auxotrophs. The transformed haploid yeast cells can then be mated or fused to give a diploid yeast cell capable of producing both the heavy and the light chains. The diploid strain is then able to secret the fully assembled and biologically active antibody. The relative expression levels of the two chains can be optimized, for example, by using vectors with different copy number, using transcriptional promoters of different strengths, or inducing expression from inducible promoters driving transcription of the genes encoding one or both chains.

In one embodiment, the respective heavy and light chains of a plurality of different anti-REG4 antibodies (the “original” antibodies) are introduced into yeast haploid cells to create a library of haploid yeast strains of one mating type expressing a plurality of light chains, and a library of haploid yeast strains of a different mating type expressing a plurality of heavy chains. These libraries of haploid strains can be mated (or fused as spheroplasts) to produce a series of diploid yeast cells expressing a combinatorial library of antibodies comprised of the various possible permutations of light and heavy chains. The combinatorial library of antibodies can then be screened to determine whether any of the antibodies has properties that are superior (e.g. higher affinity for REG4) to those of the original antibodies. See. e.g., WO2005/040395.

In another embodiment, antibodies of the present invention are human domain antibodies in which portions of an antibody variable domain are linked in a polypeptide of molecular weight approximately 13 kDa. See, e.g., U.S. Pat. Publication No. 2004/0110941. Such single domain, low molecular weight agents provide numerous advantages in terms of ease of synthesis, stability, and route of administration.

### Biological Activity of Humanized Anti-REG4 Antibodies

Antibodies having the characteristics identified herein as being desirable in a humanized anti-REG4 antibody can be screened for inhibitory biologic activity in vitro or suitable binding affinity. Agonist antibodies may be distinguished from antagonist antibodies using the biological assay provided in the Examples below. Antibodies that exhibit antagonist activity will block the activity of REG4.

To screen for antibodies that bind to the epitope on human REG4 bound by an antibody of interest (e.g., those that block binding of REG4), a routine cross-blocking assay such as that described in ANTIBODIES, A LABORATORY MANUAL, Cold Spring Harbor Laboratory, Ed Harlow and David Lane (1988), can be performed. Antibodies that bind to the same epitope are likely to cross-block in such assays, but not all cross-blocking antibodies will necessarily bind at precisely the same epitope since cross-blocking may result from steric hindrance of antibody binding by antibodies bind at overlapping epitopes, or even nearby non-overlapping epitopes.

Alternatively, epitope mapping, e.g., as described in Champe et al. (1995) J. Biol. Chem. 270:1388-1394, can be performed to determine whether the antibody binds an epitope of interest. “Alanine scanning mutagenesis,” as described by Cunningham and Wells (1989) Science 244: 1081-1085, or some other form of point mutagenesis of amino acid residues in human REG4 may also be used to determine the functional epitope for an anti-REG4 antibody of the present invention. Mutagenesis studies, however, may also reveal amino acid residues that are crucial to the overall three-dimensional structure of REG4 but that are not directly involved in antibody-antigen contacts, and thus other methods may be necessary to confirm a functional epitope determined using this method.

The epitope bound by a specific antibody may also be determined by assessing binding of the antibody to peptides comprising fragments of human REG4 (SEQ ID NO: 65). A series of overlapping peptides encompassing the sequence of REG4 may be synthesized and screened for binding, e.g. in a direct ELISA, a competitive ELISA (where the peptide is assessed for its ability to prevent binding of an antibody to REG4 bound to a well of a microtiter plate), or on a chip. Such peptide screening methods may not be capable of detecting some discontinuous functional epitopes, i.e. functional epitopes that involve amino acid residues that are not contiguous along the primary sequence of the REG4 polypeptide chain.

The epitope bound by antibodies of the present invention may also be determined by structural methods, such as X-ray crystal structure determination (e.g., WO2005/044853), molecular modeling and nuclear magnetic resonance (NMR) spectroscopy, including NMR determination of the H-D exchange rates of labile amide hydrogens in REG4 when free and when bound in a complex with an antibody of interest (Zinn-Justin et al. (1992) Biochemistry 31:11335-11347; Zinn-Justin et al. (1993) Biochemistry 32:6884-6891).

With regard to X-ray crystallography, crystallization may be accomplished using any of the known methods in the art (e.g. Giege et al. (1994) Acta Crystallogr. D50:339-350; McPherson (1990) Eur. J. Biochem. 189:1-23), including microbatch (e.g. Chayen (1997) Structure 5:1269-1274), hanging-drop vapor diffusion (e.g. McPherson (1976) J. Biol. Chem. 251:6300-6303), seeding and dialysis. It is desirable to use a protein preparation having a concentration of at least about 1 mg/mL and preferably about 10 mg/mL to about 20 mg/mL. Crystallization may be best achieved in a precipitant solution containing polyethylene glycol 1000-20,000 (PEG; average molecular weight ranging from about 1000 to about 20,000 Da), preferably about 5000 to about 7000 Da, more preferably about 6000 Da, with concentrations ranging from about 10% to about 30% (w/v). It may also be desirable to include a protein stabilizing agent, e.g. glycerol at a concentration ranging from about 0.5% to about 20%. A suitable salt, such as sodium chloride, lithium chloride or sodium citrate may also be desirable in the precipitant solution, preferably in a concentration ranging from about 1 mM to about 1000 mM. The precipitant is preferably buffered to a pH of from about 3.0 to about 5.0, preferably about 4.0. Specific buffers useful in the precipitant solution may vary and are well-known in the art. Scopes, Protein Purification: Principles and Practice, Third ed., (1994) Springer-Verlag, New York. Examples of useful buffers include, but are not limited to, HEPES, Tris, MES and acetate. Crystals may be grow at a wide range of temperatures, including 2° C., 4° C., 8° C. and 26° C.

Antibody:antigen crystals may be studied using well-known X-ray diffraction techniques and may be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al. eds., Academic Press; U.S. Patent Application Publication No. 2004/0014194), and BUSTER (Bricogne (1993) Acta Cryst. D49:37-60; Bricogne (1997) Meth. Enzymol. 276A:361-423, Carter & Sweet, eds.; Roversi et al. (2000) Acta Cryst. D56:1313-1323).

Additional antibodies binding to the same epitope as an antibody of the present invention may be obtained, for example, by screening of antibodies raised against REG4 for binding to the epitope, or by immunization of an animal with a peptide comprising a fragment of human REG4 comprising the epitope sequence. Antibodies that bind to the same functional epitope might be expected to exhibit similar biological activities, such as blocking receptor binding, and such activities can be confirmed by functional assays of the antibodies.

Antibody affinities may be determined using standard analysis. Preferred humanized antibodies are those that bind human REG4 with a K_{d} value of no more than about 1×10⁻⁷; preferably no more than about 1×10⁻⁸; more preferably no more than about 1×10⁻⁹; and most preferably no more than about 1×10⁻¹⁰ or even 1×10⁻¹¹ M.

The antibodies and fragments thereof useful in the present compositions and methods are biologically active antibodies and fragments. As used herein, the term “biologically active” refers to an antibody or antibody fragment that is capable of binding the desired the antigenic epitope and directly or indirectly exerting a biologic effect. As used herein, the term “specific” refers to the selective binding of the antibody to the target antigen epitope. Antibodies can be tested for specificity of binding by comparing binding to REG4 to binding to irrelevant antigen or antigen mixture under a given set of conditions. If the antibody binds to REG4 at least 10, and preferably 50 times more than to irrelevant antigen or antigen mixture then it is considered to be specific. An antibody that “specifically binds” to REG4 does not bind to proteins that do not comprise the REG4-derived sequences, i.e. “specificity” as used herein relates to REG4 specificity, and not any other sequences that may be present in the protein in question. For example, as used herein, an antibody that “specifically binds” to a polypeptide comprising REG4 will typically bind to FLAG®-REG4, which is a fusion protein comprising REG4 and a FLAG® peptide tag, but it does not bind to the FLAG® peptide tag alone or when it is fused to a protein other than REG4.

REG4-specific binding compounds of the present invention, such as antagonistic REG4 specific antibodies, can inhibit its biological activity in any manner, including but not limited to decreasing proliferation of cells that respond to REG4. In particular, the REG4 binding compounds will inhibit phosphorylation events of various receptor and intracellular molecules described above. The binding compounds can be subjected to the biological assays provided herein.

### Pharmaceutical Compositions

To prepare pharmaceutical or sterile compositions including REG4 antibody, the cytokine analogue or mutein, antibody thereto, or nucleic acid thereof, is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with physiologically acceptable carriers, excipients, or stabilizers in the form of, e.g., lyophilized powders, slurries, aqueous solutions or suspensions. See, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

Toxicity and therapeutic efficacy of the antibody compositions, administered alone or in combination with an immunosuppressive agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀ to ED₅₀. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

The mode of administration is not particularly important. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of antibody used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is substantially derived from the same species as the animal targeted for treatment (e.g. a humanized antibody for treatment of human subjects), thereby minimizing any immune response to the reagent.

Antibodies, antibody fragments, and cytokines can be provided by continuous infusion, or by doses at intervals of, e.g., one day, 1-7 times per week, one week, two weeks, monthly, bimonthly, etc. Doses may be provided intravenously, subcutaneously, topically, orally, nasally, rectally, intramuscular, intracerebrally, intraspinally, or by inhalation. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

As used herein, “inhibit” or “treat” or “treatment” includes a postponement of development of the symptoms associated with autoimmune disease or pathogen-induced immunopathology and/or a reduction in the severity of such symptoms that will or are expected to develop. The terms further include ameliorating existing uncontrolled or unwanted autoimmune-related or pathogen-induced immunopathology symptoms, preventing additional symptoms, and ameliorating or preventing the underlying causes of such symptoms. Thus, the terms denote that a beneficial result has been conferred on a vertebrate subject with an autoimmune or pathogen-induced immunopathology disease or symptom, or with the potential to develop such a disease or symptom.

As used herein, the term “therapeutically effective amount” or “effective amount” refers to an amount of an REG4-specific binding compound, e.g. and antibody, that when administered alone or in combination with an additional therapeutic agent to a cell, tissue, or subject is effective to prevent or ameliorate the autoimmune disease or pathogen-induced immunopathology associated disease or condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent, e.g., a cytokine, antibody, steroid, chemotherapeutic agent, antibiotic, or radiation, are well known in the art, see, e.g., Hardman et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.

Chemotherapeutic agents include receptor tyrosine kinase inhibitors, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON. toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; IGFR inhibitors; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

Typical veterinary, experimental, or research subjects include monkeys, dogs, cats, rats, mice, rabbits, guinea pigs, horses, and humans.

### Uses

The present invention provides methods for using anti-REG4 antibodies and fragments thereof for the treatment and diagnosis of proliferative disorders and conditions.

The present invention provides methods for diagnosing the presence of a cancer by analyzing expression levels of REG4 in test cells, tissue or bodily fluids compared with REG4 levels in cells, tissues or bodily fluids of preferably the same type from a control. As demonstrated herein, an increase in level of REG4 expression, for example, in the patient versus the control is associated with the presence of cancer.

Typically, for a quantitative diagnostic assay, a positive result indicating the patient tested has cancer or an infectious disease, is one in which the cells, tissues, or bodily fluids has an REG4 expression level at least two times higher, five times higher, ten times higher, fifteen times higher, twenty times higher, twenty-five times higher.

Assay techniques that may be used to determine levels of gene and protein expression, such as REG4, of the present inventions, in a sample derived from a host are well known to those of skill in the art. Such assay methods include radioimmunoassays, reverse transcriptase PCR (RT-PCR) assays, quantitative real-time PCR assays, immunohistochemistry assays, in situ hybridization assays, competitive-binding assays, western blot assays, ELISA assays, and flow cytometric assays, for example, two color FACS analysis for M2 versus M1 phenotyping of tumor-associated macrophages (Mantovani et al., (2002) TRENDS in Immunology 23:549-555).

An ELISA assay initially comprises preparing an antibodies of the present invention, specific to REG4, preferably 3C2.3D10, 4C5.3B10, 9F3.3A4, 12E1.3C11, 13E1.1B11, 40F6.3F6, 70A9.3C2, and 86C1.2B7 (collectively “REG4 antibodies”). In addition, a reporter antibody generally is prepared that binds specifically to REG4. The reporter antibody is attached to a detectable reagent such as radioactive, fluorescent or an enzymatic reagent, for example horseradish peroxidase enzyme or alkaline phosphatase.

To carry out the ELISA, at least one of the REG4 antibodies described above is incubated on a solid support, e.g., a polystyrene dish that binds the antibody. Any free protein binding sites on the dish are then covered by incubating with a non-specific protein, such as bovine serum albumin. Next, the sample to be analyzed is incubated in the dish, during which time REG4 binds to the specific REG4 antibody attached to the polystyrene dish. Unbound sample is washed out with buffer. A reporter antibody specifically directed to REG4 and linked to horseradish peroxidase is placed in the dish resulting in binding of the reporter antibody to any monoclonal antibody bound to REG4. Unattached reporter antibody is then washed out. Reagents for peroxidase activity, including a calorimetric substrate are then added to the dish. Immobilized peroxidase, linked to REG4 antibodies, produces a colored reaction product. The amount of color developed in a given time period is proportional to the amount of REG4 protein present in the sample. Quantitative results typically are obtained by reference to a standard curve.

A competition assay may be employed wherein antibodies specific to REG4 are attached to a solid support and labeled REG4 and a sample derived from the host are passed over the solid support and the amount of label detected attached to the solid support can be correlated to a quantity of REG4 in the sample.

The above tests may be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. The term “blood” is meant to include whole blood, plasma, serum or any derivative of blood.

The broad scope of this invention is best understood with reference to the following examples, which are not intended to limit the inventions to the specific embodiments. The specific embodiments described herein are offered by way of example only, and the invention is to be limited by the terms of the appended claims, along with the full scope of equivalents to which such claims are entitled.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### Example 2

### Humanization of Anti-human REG4 Antibodies

The humanization of antibodies is described generally, e.g., in PCT patent application publications WO 2005/047324 and WO 2005/047326.

Briefly, the amino acid sequence of the non-human VH domain (e.g. SEQ ID NOs: 1-8) is compared to a group of five human VH germline amino acid sequences; one representative from subgroups IGHV1 and IGHV4 and three representatives from subgroup IGHV3. The VH subgroups are listed in M.-P. Lefranc (2001) “Nomenclature of the Human Immunoglobulin Heavy (IGH) Genes”, Experimental and Clinical Immunogenetics 18:100-116. The framework sequences of the human germline sequence with the closest match are used to construct a humanized VH domain.

The rodent anti-huREG4 antibodies disclosed herein are all of the kappa subclass of VL. The amino acid sequences of the non-human VL domain (e.g. SEQ ID NOs: 9-16) is compared to a group of four human VL kappa germline amino acid sequences. The group of four is comprised of one representative from each of four established human VL subgroups listed in V. Barbie & M.-P. Lefranc (1998) “The Human Immunoglobulin Kappa Variable (IGKV) Genes and Joining (IGKJ) Segments”, Experimental and Clinical Immunogenetics 15:171-183 and M.-P. Lefranc (2001) “Nomenclature of the Human Immunoglobulin Kappa (IGK) Genes”, Experimental and Clinical Immunogenetics 18:161-174. The four subgroups also correspond to the four subgroups listed in Kabat et al. (1991-5th Ed.) “Sequences of Proteins of Immunological Interest”, U.S. Department of Health and Human Services, NIH Pub. 91-3242, pp. 103-130. The framework sequences of the human germline sequence with the closest match are used to construct a humanized VL domain.

Once the target amino acid sequences of the variable heavy and light chains are determined, plasmids encoding the full-length humanized antibody may be generated. Plasmid sequences may be altered using Kunkel mutagenesis (see, e.g., Kunkel T A. (1985) Proc. Natl. Acad. Sci. U.S.A 82:488-492) to change the DNA sequence to the target humanized antibody sequences. Simultaneously, codon optimization may be performed to provide for potentially optimal expression.

Antibodies of the present invention can be humanized using a method that identifies an acceptor germline sequence for a humanized antibody, and comprises the steps of: a) identifying a non-human antibody that has the desired biological activity; b) determining the amino acid sequence of a non-human antibody V_{H} and V_{L} domains; and c) comparing the nonhuman antibody sequence to a group of human germline sequences, wherein the comparison comprises the substeps of: 1) assigning the non-human V sequences residue numbers according to Kabat supra; 2) delineating the CDR and FR regions in the sequence according to Kabat supra; 3) assigning a predetermined numerical score at specific residue position for which the non-human and human antibody germline sequences are identical; and 4) totaling all of the residue scores to generate a total score for each human germline sequence; and d) identifying the human germline sequence with the highest total residue score as the acceptor germline sequence. In one embodiment, the method further comprises the substeps of: 5) assigning a numerical score of 1 for each FR residue position for which the non-human and human antibody germline sequences are identical that was not scored in substep (3) to germline sequences with identical total residue scores after substep (4); 6) totaling all of the residue scores to generate a total score for each human germline sequence. In a specific embodiment, the non-human antibody is specific for REG4 and enhances the biological activity of REG4. Also provided herein is an antibody generated by the above method.

In one embodiment, the REG4 antibody is humanized using the following method. First, the non-human V_{L} and V_{H} domains of the REG4 antibody are cloned and sequenced, and the amino acid sequence determined. Then, the non-human V_{H} sequence are compared to a group of three human V_{H} germline amino acid sequences. The three groups contain one representative from each of subgroups IGHV1, IGHV3 and IGHV4. The V_{H} subgroups are listed in M.-P. Lefranc, Exp. Clin. Immunogenetics, 18:100-116 (2001). Specifically, the comparison with the three germline sequences begins with the assignment of residue numbers to the non-human V_{H} sequence according to the Kabat numbering system. See Kabat, et al., U.S. Department of Health and Human Services, NIH Pub. 91-3242 (5th Ed., 1991). The non-human V_{H} sequence are then aligned with each of the three human germline sequences. Since the V genes only comprise V_{H} residues 1-94, only these residues are considered in the alignment. Next, the complementarity-determining (CDR) and framework (FR) regions in the sequence are delineated. CDR and FR are delineated according to the combination of the definitions provided in Kabat, et al., U.S. Department of Health and Human Services, NIH Pub. 91-3242 (5th Ed., 1991), and C. Chothia & A. M. Lesk, J. Mol. Biol., 196:901-917 (1987). Therefore, the CDR definition used is residues 26-35 for CDR1, residues 50-65 for CDR2, and CDR3 is residues 95-102 for CDR3 of the V_{H} domain. The next step involves assigning a numerical score at identified residue position where the non-human and human sequences are identical. One example of this scoring is shown in Table 4 below.

TABLE 4

| Residue # | Score | Reason |
|---|---|---|
| 24 | 3 | Affects CDR-H1 |
| 27 | 4 | Affects CDR-H1,3* |
| 29 | 4 | Affects CDR-H1* |
| 34 | 4 | Affects CDR-H1* |
| 35 | 2 | VH/VL interface |
| 37 | 2 | VH/VL interface |
| 48 | 3 | Affects CDR-H2 |
| 49 | 3 | Affects CDR-H2 |
| 50 | 2 | VH/VL interface |
| 58 | 2 | VH/VL interface |
| 60 | 2 | VH/VL interface |
| 63 | 3 | Affects CDR-H2 |
| 67 | 3 | Affects CDR-H2 |
| 69 | 3 | Affects CDR-H2 |
| 71 | 4 | Affects CDR-H2* |
| 73 | 3 | Affects CDR-H1 |
| 76 | 3 | Affects CDR-H1 |
| 78 | 3 | Affects CDR-H1 |
| 94 | 4 | Affects CDR-H3* |
| max | 57 | |

*Noted as affecting CDR conformation in C. Chothia et al., Nature 342: 877-883, (1989).

After the residue positions are assigned a numerical score, all of the residue scores are totaled. The acceptor germline sequence is the one with the highest total score. In a case where two or more germline sequences have identical scores, then add 1 to the total for each position where the non-human and human sequences are IDENTICAL for the following FR residues: 1-23, 25, 36, 38-47, 66, 68, 70, 72, 74, 75, 77, and 79-93 (max 60). The residue scores are totaled again, and the acceptor germline sequence is the one with the highest total score. If two or more germline sequences still have identical scores, either one can be used as the acceptor germline sequence.

If the V_{L} sequence is a member of the kappa subclass of V_{L}, the non-human V_{L} sequence from the REG4 specific antibody is compared to a group of four human V_{L} kappa germline amino acid sequences. The four sequences are comprised of one representative from each of four established human V_{L} subgroups listed in V. Barbie & M.-P. Lefranc, Exp. Clin. Immunogenetics 15:171-183 (1998) and M.-P. Lefranc, Exp. Clin. Immunogenetics 18:161-174 (2001). The four sequences also correspond to the four subgroups listed in Kabat et al., U.S. Department of Health and Human Services, NIH Pub. 91-3242, pp. 103-130 (5th Ed., 1991). The comparison of the non-human sequence to the four germline sequences begins with the assignment of residue numbers to the non-human V_{L} sequence residues according to Kabat et al., U.S. Department of Health and Human Services, NIH Pub. 91-3242 (5th Ed., 1991). The non-human V_{L} sequences are then aligned with each of the four human germline sequences. Since the V genes only comprise V_{L} residues 1-95, only these residues are considered in the alignment. Next, the complementarity-determining (CDR) and framework (FR) regions are delineated in the sequence. CDR and FR are delineated according to the combination of the definitions provided in Kabat et al., U.S. Department of Health and Human Services, NIH Pub. 91-3242 (5th Ed. 1991), and C. Chothia & A. M. Lesk, J. Mol. Biol., 196:901-917 (1987). Therefore, the CDR definition used is residues 24-34 for CDR1, residues 50-56 for CDR2, and residues 89-97 for CDR3 of the V_{L} domain. The next step involves assigning a numerical score at identified residue position where the non-human and human sequences are identical. One example of this scoring is shown in Table 5 below.

TABLE 5

| Residue # | Score | Reason |
|---|---|---|
| 2 | 4 | Affects CDR-L1,3* |
| 25 | 4 | Affects CDR-L1* |
| 29 | 4 | Affects CDR-L1,3* |
| 34 | 2 | VL/VH interface |
| 43 | 2 | VL/VH interface |
| 55 | 2 | VL/VH interface |
| 58 | 3 | Affects CDR-L2 |
| 89 | 2 | VL/VH interface |
| 91 | 2 | VL/VH interface |
| 94 | 2 | VL/VH interface |
| max | 27 | |

*Noted as affecting CDR conformation in C. Chothia et al., Nature 342: 877-883, (1989).

After the residue positions are assigned a numerical score, all of the residue scores are totaled. The acceptor germline sequence is the one with the highest total score. In a case where two or more germline sequences have identical scores, then add 1 to the total for each position where the non-human and human sequences are IDENTICAL for the following FR residues: 1-3, 5-23, 35-42, 44-49, 57, 59-88 (max 67). The residue scores are totaled again, and the acceptor germline sequence is the one with the highest total score. If two or more germline sequences still have identical scores, either one can be used as the acceptor germline sequence.

### Example 3

### Determining the Equilibrium Dissociation Constant (K_{d}) for Anti-Human REG4 Antibodies Using KinExA Technology

The equilibrium dissociation constants (K_{d}) for anti human REG4 antibodies are determined using the KinExA 3000 instrument. Sapidyne Instruments Inc., Boise Id., USA. KinExA uses the principle of the Kinetic Exclusion Assay method based on measuring the concentration of uncomplexed antibody in a mixture of antibody, antigen and antibody-antigen complex. The concentration of free antibody is measured by exposing the mixture to a solid-phase immobilized antigen for a very brief period of time. In practice, this is accomplished by flowing the solution phase antigen-antibody mixture past antigen-coated particles trapped in a flow cell. Data generated by the instrument are analyzed using custom software. Equilibrium constants are calculated using a mathematical theory based on the following assumptions:

1. The binding follows the reversible binding equation for equilibrium:

kₒₙ [Ab] [Ag]=k_{off}[AbAg]

2. Antibody and antigen bind 1:1 and total antibody equals antigen-antibody complex plus free antibody.

3. Instrument signal is linearly related to free antibody concentration.

PMMA particles (Sapidyne, Cat No. 440198) are coated with biotinylated REG4 (or a fragment thereof, such as the extracellular domain) according to Sapidyne “Protocol for coating PMMA particles with biotinylated ligands having short or nonexistent linker arms.” EZ-link TFP PEO-biotin (Pierce, Cat. No. 21219) is used for biotinylation of REG4, as per the manufacturer's recommendations (Pierce bulletin 0874).

### Example 4

### Determining the Equilibrium Dissociation Constant (K_{d}) for Humanized Anti-Human REG4 Antibodies Using BIAcore Technology

BIAcore determinations are performed essentially as described at Example 4 of co-pending, commonly assigned U.S. patent application Ser. No. 11/511,635 (filed 29 Aug. 2006). Briefly, ligands (anti-REG4-1-hIg) are immobilized on a BIAcore CM5 sensor chip using standard amine-coupling procedure. Kinetic constants for the various interactions are determined using BIAevaluation software 3.1. The K_{d} is determined using the calculated dissociation and association rate constants.

The kinetic binding activity of Rat Anti-REG4_H antibodies against Schering-Plough BioPharma REG4_H, lot 04ABY, was measured by surface plasmon resonance using a Biacore T100 system (Biacore, GE Healthcare, Piscataway, N.J.). Approximately 30 RUs of REG4 was immobilized via amine coupling chemistry onto a Series S Sensor Chip CM5 (Research grade, BR-1006-68). HBS-EP+ buffer (BR-1006-69) was used as the running buffer with a flow rate of 304/min. Schering-Plough BioPharma Rat Anti-REG4_H antibodies were injected over the immobilized REG4_H surface at varying concentrations, ranging from [0.091 nM to 600 nM], at a flow rate of 30 μL/min. Following each injection cycle, the CM5 chip surface was regenerated using a 10 mM Glycine pH 1.5 solution followed by an injection of 25 mM NaOH solution at a flow rate of 75 μL/min.

Background subtraction binding sensorgrams were used for analyzing the rate constants; association (kₐ), dissociation (k_{d}), and the equilibrium dissociation constant K_{D}. The resulting data sets were fitted with a Bivalent Analyte Model using the Biacore T100 evaluation software (version 1.1.1).

REG4 antibodies 3C2.3D10, 9F3.3A4, 12E1.3C11, 13E1.1B11, 70A9.3C2 had the following Kd values:

TABLE 6
Affinity measurements of REG4 antibodies

| Analyte | | Ka (1/Ms) | Kd (1/s) | Apparent |
|---|---|---|---|---|
| (mAb) | Capture antigen | (×10⁵) | (×10⁻⁶) | Kd (pM) |
| 3C2.3D10 | hREG4 protein | 0.76 | 18 | 240 |
| 9F3.3A4 | hREG4 protein | 0.66 | 3 | 3542 |
| 12E1.3C11 | hREG4 protein | 3.30 | 21 | 62 |
| 13E1.1B11 | hREG4 protein | 0.04 | 1171 | 316351 |
| 70A9.3C2 | hREG4 protein | 16.75 | 1006 | 601 |

### Example 5

### Cell Lines

Human colon cancer cell lines KM12, HCT116 and HT29 from NCI 60 panel from NCI. Human prostate cancer cell lines PC3 was purchased from ATCC. KM12 and PC3 cells were maintained as monolayers in RPMI supplemented with 10% FCS and 2 mmol/L of L-glutamine, sodium pyruvate and non-essential amino acids. HCT116 and HT29 cells were maintained as monolayers in DMEM supplemented with 10% FCS and 2 mmol/L of L-glutamine, sodium pyruvate and non-essential amino acids. The cells were grown at 37° C. in a humidified atmosphere with 5% CO₂.

### Example 6

### siRNA Knockdown

Cells were plated at a density of 1.0-2.0×10⁵ cells/mL in 6-well plates. Cells were transfected with in OptiMEM media (Gibco 31985) containing 100 nM siRNA using Dharmafect 3 transfection ket (Dharmacon T-2003) for KM12 cells or Dharmafect 4 (Dharmacon T-2004) for PC3 cells for 24 hours according to the manufacturer's protocol. Invitrogen REG4 siRNA (pooled REG4HSS130525, REG4HSS130526, REG4HSS130527), Invitrogen stealth negative control (pooled 12935-200, 12935-300, 12935-400), KSP positive control (Dharmacon custom WANWK-000001) or Dharmacon negative control siRNA (On target Plus Duplex, Dharmacon, D-001810-10) were used. Confirmation of knockdown was assessed at the mRNA level by Taqman and protein levels by western blot.

### Example 7

### Cell Titer Glo (“CTG”) Viability Assay

Twenty-four hours after transfection, 3×10³ cells were plated in black view plates and incubated at 37° C. in a humidified atmosphere with 5% CO2. Cell Titer Glo reagent (Promega G7573) was added according to the manufacturer's directions 72-96 h post-transfection. Briefly, 100 uL/well of Cell Titer Glo reagent was added and the contents were mixed on the plate shaker at 400 rpm for 2 minutes to induce lysis. After a 10 minute incubation, the luminescence was read with a multiwell plate reader (DTX 880 multimode reader) and integration time of 100,000 μs, using the method of fluorescence intensity.

### Example 8

### MTT Cell Proliferation Assay

At the end of assay, 10 ul MTT assay reagent (Roche, 11 465 007 001) was added to wells according to the manufacturer's directions. Four hours later, 100 ul of solubilization solution was added to plates and plates were incubated overnight in 37° C. incubator in a humidified atmosphere. The absorbance of A550 nm-A690 nm was measured with an ELISA plate reader (Molecular Devices Spectra max 340PC).

### Example 9

### Soft Agar Colony Formation Assay

96-well flat bottom plates (Costar 3474) were coated with 1% agarose (Cambrex 50070). Twenty-four hours after transfection, 5 to 10×10² cells/well were added in media with a final concentration of 0.33% agarose. Wells were coated with 100 μL media upon agarose solidification. After incubation for 7 days at 37° C. in a humidified atmosphere with 5% CO2, 29 μL of Alamar blue (Biosource DAL 1100) was added to each well. The fluorescence was read 4, 8, and 24 hours after the addition of Alamar blue using a multiwell plate reader and a Rhodamine filter excitation 530 nm and emission 590 nm.

### Example 10

### Spheroid Assay

Round bottom 96-well plates were coated with Polyhema (Sigma P3932). Twenty-four hours after transfection, cells were plated at a density of 1×10⁴ cells/well in media. Cells were grown on a waver platform to allow spheroid formation at 37° C. in a humidified atmosphere with 5% CO₂ for 7 days. The supernatant was transferred to a black assay plate and the spheroids lysed in a final concentration of 2% Triton X-100. The Promega LDH kit (G7890) was used according to the manufacturer's directions for the supernatant and lysed spheroids to measure cell death and intracellular LDH from spheroids, respectively. Briefly, 100 μL LDH reagent was added, shaked 30 seconds and incubate 10 minutes before reading fluorescence on a plate reader at 560/590 nm.

### Example 11

### Cell Cycle Analysis by BrdU Incorporation

Cell cycle analysis was determined by transfecting cells with siRNA for 24-72 h and harvesting (adherent and suspension) cells after being pulsed for 30 minutes with 10 μM BrdU. Cells were fixed in 70% ethanol and stored at −20° C. overnight. Cells were centrifuged, resuspended in 0.1M HCl in PBS-0.5% Triton X-100 and incubated on ice for 10 minutes. Cells were washed with dH₂0, resuspended in dH₂0 and boiled for 10 minutes, followed by 10 minutes on ice. Cells were then washed with PBS-0.5% Triton X-100 and resuspended in 10 uL 0.1% BSA, 20 uL anti-BrdU-FITC (BD 347583) and 70 uL PBS. After 30 minutes, cells were washed and resuspended in propidium iodide-RNase solution (BD Pharmingen 550825). The cells were then analyzed for cell cycle perturbation using a FACSCanto (Becton Dickinson). The FlowJo program was used to quantitate the distribution of cells in each cell cycle phase: sub-G₁ (apoptotic cells), G₁, S, and G₂-M.

### Example 12

### Detection of Cell Death by Annexin V-Alexa Fluor 488/Propidium Iodide Staining

Cell death was assessed by transfecting KM12 and PC3 cells plated at a density of 1×10⁵ cells/6-well plate with REG4 siRNA for 96 h and then stained with Annexin V-Alexa Fluor 488 (Invitrogen A13201) as described in the manufacturer's protocol. Briefly, floating and adherent cells were collected using cell dissociation buffer (Invitrogen 13151) and cells were incubated in 85 μL Annexin V binding buffer (BD Pharmingen 556565), 5 μL Annexin V-Alexa Fluor 488 and 10 μL propidium iodide (BD Pharmingen 550825) at 37° C. in the dark. After 15 minutes, 400 μL of Annexin V binding buffer was added and samples were analyzed within 30 minutes by flow cytometry using a FACSCanto. FACS plots were analyzed in FlowJo to differentiate between apoptotic and necrotic cells based on Annexin V and PI staining.

### Example 13

### Detection of Single Strand Breaks by F7-26 Staining

Detection of apoptosis was determined by transfecting KM12 and PC3 cells plated at a density of 1×10⁵ cells/6-well plate with REG4 siRNA for 96 h and then floating and adherent cells were collected using 0.05% trypsin (Sigma 59417C). Cells were fixed in 100% methanol overnight. Cells were then resuspended in 250 uL formamide and incubated at 75 degrees for 15 minutes. Cells were blocked for 15 minutes in 1% milk in PBS, probed with 1:10 dilution of F7-26 antibody (Chemicon International MAB3299) for 15 minutes. Samples were then washed with PBS and probed for 15 minutes with a 1:50 dilution of anti-mouse IgM (Chemicon International AP128F) for 15 minutes in the dark. Cells were resuspended in PI/RNase solution and analyzed using a FACSCanto. FACS plots were analyzed in FlowJo to differentiate between normal and apoptotic cells based on F7-26 staining.

Table 7 summarizes the effects of REG4 siRNA knockdown in the assays noted above.

| | | | | Anchorage | | | | |
|---|---|---|---|---|---|---|---|---|
| | Growth on Plastic | | | Independent | | Cell Death | | |
| | CTG/ | | cell | | Spher- | Caspase | strand | Annexin |
| Assay: | LDH | CFSE | cycle | SAA | oid | 3/7 | breaks | V |
| KM12 | 12/14 | nd | | 1/3 | 3/3 | 0/4 | 1/1 | 1/1 |
| PC3 | 11/12 | 2/2 | 3/3 | 4/4 | 3/3 | 0/2 | 3/3 | 2/2 |
| AGS | 1/1 | nd | nd | nd | nd | nd | nd | nd |

### Example 14

### Western Blot Analysis

Preparation of Protein Lysates was Done Using the Ripa Buffer (Sigma) containing phosphatase and protease inhibitors according to the manufacturer's directions. Western blot analyses were done in 1-3% BSA-0.05% TBS-Tween at a dilution of 0.1-0.2 μg/ml using antibodies directed against REG4 (R&D System AF1379), p-Akt/total Akt (Cell Signaling 9271/9272), p-EGFR/total EGFR (Cell Signaling 2234/2232), Bcl2 (Cell Signaling 2872), E2F1 (Cell Signaling 3742), c-Myc (Cell Signaling 9402), Bcl-XL (Cell Signaling 2762), PUMA (Cell Signaling 4976), p-Hsp27/total Hsp27 (Cell Signaling 2401/Santa Cruz Biotech sc-13132) overnight at 4° C. Secondary conjugated sheep anti-mouse IgG (Amersham NA931) and donkey anti-rabbit IgG (Amersham NA934V) HRP antibodies were used at a concentration of 1:5000 at room temperature for an hour. Blots were developed using pico, dura or femto chemiluminescent reagents from Pierce (34076, 34080, 34095).

Media was collected 48 h after transfection and centrifuged to remove cell debris. Reg IV was immunoprecipitated from 2 mL samples with Protein A/G beads, 40 ug mouse anti-Reg IV (R&D) or isotype control and overnight incubation. IP samples were run by Western blot, as described above.

### Example 15

### Exogenous REG4 Induced Growth/REG4 Antibody Screening

96-well MaxiSorp plate (Nunc 12-565-136) were sterilized under UV for 1 hour. Plates were coated with recombinant REG4 purchased from R&D System (1379-RG-50) or produced in house at 1-20 μg/ml in 50 μl DMEM medium at room temperature overnight in a moisturized chamber. 7×10³HCT116 cells in 50 ul DMEM medium with 1% FBS were plated to wells with and without 10-20 μg/ml anti-REG4 antibodies (e.g., 3C2.3D10, 4C5.3B10, 9F3.3A4, 12E1.3C11, 13E1.1B11, 40F6.3F6, 70A9.3C2, and 86C1.2B7) to a final 0.5% FBS concentration. MTT cell proliferation assays were performed after cells had been incubated in 37° C. tissue culture incubator for 45-48 hr.

### Example 16

### Anti-REG4 Activity in Inhibition of Endogenous REG4

PC3 and KM12 cells were transfection stressed for 24 h with 50 nM pooled negative control siRNA from Invitrogen. Cells were re-plated in 96 well plates (BD 3072) at 1000 cells per well. REG4 antibodies were added at a final concentration of 5-50 μg/ml in 1% FBS RPMI and plates were incubated at 37° C. in a humidified chamber with 5% CO₂ for 5 days with an exchange of 10 μg/well antibodies 48 h after prior antibody treatment. On day 5, a MTT assay was performed according to the kit directions.

### Example 17

### Exogenous Reg4 Induced MAPK Pathway Gene Activation

HCT116 cells plated overnight in tissue culture dishes were washed once with serum free DMEM and serum starved in serum free DMEM for 24 h at 37° C. in a humidified chamber with 5% CO₂. 500 nM recombinant Reg4 expressed in house in serum free DMEM was used to stimulate the cells by incubation at 37° C. in a humidified chamber with 5% CO₂ for 5 minutes, 30 minutes and 2 hours. At the end of stimulation, cells were washed once with PBS and lysed with RIPA buffer (Sigma) containing phosphatase and protease inhibitors according to the manufacturer's directions. 200 ug of cell lysate was applied to MAPK arrays (R&D System, ARY002) and followed by incubation of detection antibody cocktail and Streptavidin-HRP as recommended by manufacture. Phospho-MAP kinase signals were detected on X-ray film after arrays were exposed to chemiluminescent reagent (PIERCE, SuperSignal WestPico).

### Example 18

### Exogenous REG4 Induction of RTK Activation

HCT116 cells plated overnight in tissue culture dishes were washed once with serum free DMEM and serum starved in serum free DMEM for 24 h at 37° C. in a humidified chamber with 5% CO₂. 500 nM recombinant REG4 expressed in house in serum free DMEM was used to stimulate the cells by incubation at 37° C. in a humidified chamber with 5% CO₂ for 5 minutes, 30 minutes and 2 hours. At the end of stimulation, cells were washed once with PBS and lysed with RIPA buffer (Sigma) containing phosphatase and protease inhibitors according to the manufacturer's directions. 300 ug of cell lysate was applied to RTK arrays (R&D System, ARY001) and followed by incubation of detection antibody as recommended by manufacture. Phospho-RTK signals were detected on X-ray film after arrays were exposed to chemiluminescent reagent (PIERCE, SuperSignal WestPico).

### Example 19

### Exogenous REG4 Induction of p-Akt

### Discovery-1 Detection

HCT116 cells were plated on collagen pre-coated 96-well black plates (Sigma, S3190-5EA) and incubated overnight at 37° C. in a humidified chamber with 5% CO₂. Cells were washed once with serum free DMEM and serum starved in serum free DMEM for 24 hours. 100 nM to 500 nM recombinant REG4 expressed in serum free DMEM was used to stimulate the cells by incubation at 37° C. in a humidified chamber with 5% CO₂ for 5 minutes, 30 minutes and 2 hours. At the end of stimulation, cells were fixed in 3% paraformaldehyde (Alfa Acesar, 30525-89-4) at room temperature for 15 minutes.

After one wash with PBS, cells were permeabilized in cold methanol for 10 minutes in −20° C. and blocked with 3% BSA, 0.3% Triton X-100 in PBS at room temperature for 1 hour. Cells then were incubated with phospho-Akt antibody (S473; Cell Signaling Tech, #4060) diluted in blocking buffer overnight at 4° C. After 3 washes by soaking the cells in PBS for 10 minutes each, the secondary antibody, Alexa 488-Goat anti-rabbit (Invitrogen) is added at a dilution of 1 to 500 in blocking buffer and the cells were incubated at room temperature in dark for 2 hours. Cells were washed 3 times again by soaking in PBS for 10 minutes each, followed by propidium iodide staining with Propidium Iodide/RNase A solution (Becton-Dickenson) at room temperature for 10 minutes. Stained cells were covered with DABCO mounting medium (Sigma, D2522) to prevent cell lose and color diffusion. Fluorescence image capture and intensity detection was acquired by using Discovery-1 detection system (Molecular Devices) and images were analyzed with a journal written with version 6.1 Discovery-1 software with help from Molecular Devices technique support. Phospho-Akt green fluorescence intensity induced by Reg4 was compared to that of untreated cells after normalized with nuclei counts (fluorescence in red) in the images captured.

### Example 20

### Exogenous REG4 Induction of p-Akt

### MSD Detection

HCT116 cells plated overnight in tissue culture dishes were washed once with serum free DMEM and serum starved in serum free DMEM for 24 h at 37° C. in a humidified chamber with 5% CO₂. 100 nM to 500 nM recombinant Reg4 expressed in house in serum free DMEM was used to stimulate the cells by incubation at 37° C. in a humidified chamber with 5% CO₂ for 5 minutes, 30 minutes and 2 hours. At the end of stimulation, cells were washed once with PBS and lysed with RIPA buffer (Sigma) containing phosphatase and protease inhibitors according to the manufacturer's directions. 1 ug to 20 ug of cell lysate was applied to MULTI-SPOT Phospho (Ser 473)/Total Akt Assay plate (Meso Scale Discovery, K15100D-1). After a short spin at low speed, plate was incubated at room temperature for 2 hour with shaking Washes, preparation of detection antibody and preparation of Read Buffer were performed according to manufacture's suggestions. Plate was analyzed with SECTOR Imager 6000 and the percentage of phospho-Akt signal was calculated as manufacture recommended ((2*phosphor Akt signal)/(phosphor Akt signal+total Akt signal))*100.

### Example 21

### MSD Phospho-Akt Detection of Endogenous REG4 Expressing Cells

5-10 μg of KM12 and PC3 cell lysate were applied to MULTI-SPOT Phospho (Ser 473)/Total Akt Assay plate (Meso Scale Discovery, K15100D-1) following REG4 and negative control siRNA knock down for 24 hours, 48 hours and 72 hours. After a short spin at low speed, plate was incubated at room temperature for 2 hour with shaking Washes, preparation of detection antibody and preparation of Read Buffer were performed according to manufacture's suggestions. Plate was analyzed with SECTOR Imager 6000 and the percentage of phospho-Akt signal was calculated as manufacture recommended.

Table 8 summarizes the intracellular and cell surface molecules affected by REG4 siRNA knockdown in KM12 and PC3 cell lines, or REG4 treatment of HCT116 cells.

| Readout | MAPK Array | RTK Array | Western Blot |
|---|---|---|---|
| KM12 | p-GSKa/b | p-IR | Bcl-2 |
| | p-Akt | p-IGF-1R | c-Fos |
| | | | E2F-1 |
| | | | Cyclin D1 |
| PC3 | p-GSKa/b | p-IR | Bcl-2 |
| | p-Akt | p-IGF-1R | Cyclin D1 |
| | p-Hsp27 | p-EGFR | p-Hsp27 |
| | p-RSK1 | | Hsp27 |
| | p-MSK2 | | |
| | p-70 S6 kinase | | |
| HCT116 | p-ERK1 | p-IR | p-ERK |
| | p-ERK2 | p-IGF1R | Bcl-2 |
| | p-GSKa/b | p-VEGFR | c-Fos |
| | p-Akt | p-HGFR | E2F-1 |
| | p-Hsp27 | | Cyclin D1 |
| | p-MSK2 | | |
| | p-p70 S6 kinase | | |
| | pRSK1 | | |

Treatment with REG4 protein resulted in an increase of phosphorylated proteins. Conversely, siRNA REG4 knockdown in PC3 and KM12 cells resulted in a decrease of phosphorylation.

### Example 22

### Endothelial Cell Tubule Formation Assay

Microtiter plates were coated with 50 μA of ECMatrix provided as part of the In vitro Angiogenesis assay kit (Chemicon ECM625). Harvested HUVEC cells (Lonza) as per supplier's instructions in 25% EGM media and prepared a cell suspension containing 20,000 cell/100 μl media. Added 100 μl cell suspension per well. Added 50 μl 3× concentrations of proteins in 25% EGM to each well. After 18 hrs cells were stained with the MTT kit (Roche). 20 ul MTT reagent was added to the well and plates were incubated for 3 hours at 37° C. Images taken with a 4× objective using a Spot camera. 4 images were taken for each well at 4× magnification using Discovery-1. Images for each condition (12 images) were analyzed using the Angiogenesis module provided with the Metamorph software version 6.1r6.

Endothelial cell tubule formation increased with REG4 protein treatment, in a dose dependent manner (see Table 9).

Table 9 summarizes microtubule growth with REG4 treatment of HUVEC cells.

| | Avg Total | Std dev |
|---|---|---|
| REG4 Concentration | Tubule Length | Total Tubule Length |
| 2.6 μg/ml | 7613.74 | 2310.02 |
| 0.26 μg/ml | 4391.87 | 1458.88 |
| Control (25% EGM) | 3452.45 | 1443.31 |

### Example 23

### Statistical Analysis

A two-tailed Student's t test was used for statistical analysis of comparative data using the GraphPad Prism program. Data were expressed as means of at least three independent experiments ±SE, with P<0.05 considered statistically significant.

Table 10 provides a brief description of the sequences in the sequence listing.

| SEQ ID NO.: | Description |
|---|---|
| 1 | 3C2.3D10 VH |
| 2 | 4C5.3B10 VH |
| 3 | 9F3.3A4 VH |
| 4 | 12E1.3C11 VH |
| 5 | 13E1.1B11 VH |
| 6 | 40F6.3F6 VH |
| 7 | 70A9.3C2 VH |
| 8 | 86C1.2B7 Vh |
| 9 | 3C2.3D10 VL |
| 10 | 4C5.3B10 VL |
| 11 | 9F3.3A4 VL |
| 12 | 12E1.3C11 VL |
| 13 | 13E1.1B11 VL |
| 14 | 40F6.3F6 VL |
| 15 | 70A9.3C2. VL |
| 16 | 86C1.2B7 VL |
| 17 | 3C2.3D10 CDRH1 |
| 18 | 4C5.3B10 CDRH1 |
| 19 | 9F3.3A4 CDRH1 |
| 20 | 12E1.3C11 CDRH1 |
| 21 | 13E1.1B11 CDRH1 |
| 22 | 40F6.3F6 CDRH1 |
| 23 | 70A9.3C2. CDRH1 |
| 24 | 86C1.2B7 CDRH1 |
| 25 | 3C2.3D10 CDRH2 |
| 26 | 4C5.3B10 CDRH2 |
| 27 | 9F3.3A4 CDRH2 |
| 28 | 12E1.3C11 CDRH2 |
| 29 | 13E1.1B11 CDRH2 |
| 30 | 40F6.3F6 CDRH2 |
| 31 | 70A9.3C2 CDRH2 |
| 32 | 86C1.2B7 CDRH2 |
| 33 | 3C2.3D10 CDRH3 |
| 34 | 4C5.3B10 CDRH3 |
| 35 | 9F3.3A4 CDRH3 |
| 36 | 12E1.3C11 CDRH3 |
| 37 | 13E1.1B11 CDRH3 |
| 38 | 40F6.3F6 CDRH3 |
| 39 | 70A9.3C2 CDRH3 |
| 40 | 86C1.2B7 CDRH3 |
| 41 | 3C2.3D10 CDRL1 |
| 42 | 4C5.3B10 CDRL1 |
| 43 | 9F3.3A4 CDRL1 |
| 44 | 12E1.3C11 CDRL1 |
| 45 | 13E1.1B11 CDRL1 |
| 46 | 40F6.3F6 CDRL1 |
| 47 | 70A9.3C2 CDRL1 |
| 48 | 86C1.2B7 CDRL1 |
| 49 | 3C2.3D10 CDRL2 |
| 50 | 4C5.3B10 CDRL2 |
| 51 | 9F3.3A4 CDRL2 |
| 52 | 12E1.3C11 CDRL2 |
| 53 | 13E1.1B11 CDRL2 |
| 54 | 40F6.3F6 CDRL2 |
| 55 | 70A9.3C2 CDRL2 |
| 56 | 86C1.2B7 CDRL2 |
| 57 | 3C2.3D10 CDRL3 |
| 58 | 4C5.3B10 CDRL3 |
| 59 | 9F3.3A4 CDRL3 |
| 60 | 12E1.3C11 CDRL3 |
| 61 | 13E1.1B11 CDRL3 |
| 62 | 40F6.3F6 CDRL3 |
| 63 | 70A9.3C2. CDRL3 |
| 64 | 86C1.2B7 CDRL3 |
| 65 | Human REG4 Polypeptide |

What is claimed is:

1. A binding compound that binds to human REG4 comprising:
a) an antibody light chain variable domain, or antigen binding fragment thereof, having one or more CDR sequence selected from the group consisting of SEQ ID NOs: 41-64; and
b) an antibody heavy chain variable domain, or antigen binding fragment thereof, having one or more CDR sequence selected from the group consisting of SEQ ID NOs: 17-40.

2. The binding compound of claim 1 comprising:
a) an antibody light chain variable domain, or antigen binding fragment thereof, having two or more CDR sequences selected from the group consisting of SEQ ID NOs: 41-64; and
b) an antibody heavy chain variable domain, or antigen binding fragment thereof, having two or more CDR sequences selected from the group consisting of SEQ ID NOs: 17-40.

3. The binding compound of claim 2 comprising:
a) an antibody light chain variable domain, or antigen binding fragment thereof, having three CDR sequences selected from the group consisting of SEQ ID NOs: 41-64 and
b) an antibody heavy chain variable domain, or antigen binding fragment thereof, having three CDR sequences selected from the group consisting of SEQ ID NOs: 17-40.

4. A binding compound that binds to human REG4 comprising:
a) a light chain variable domain, or antigen binding fragment thereof, having at least one CDRL1 from the group consisting of SEQ ID NOs: 41-48; at least one CDRL2 from the group consisting of SEQ ID NOs: 49-56; and at least one CDRL3 from the group consisting of SEQ ID NOs: 57-64; and
b) a heavy chain variable domain, or antigen binding fragment thereof, having at least one CDRH1 from the group consisting of SEQ ID NOs: 17-24; at least one CDRH2 from the group consisting of SEQ ID NOs: 25-32; and at least one CDRH3 from the group consisting of SEQ ID NOs: 33-40.

5. A binding compound that binds to human REG4 comprising:
a) an antibody light chain variable domain of SEQ ID NO: 9, and an antibody heavy chain variable domain of SEQ ID NO: 1;
b) an antibody light chain variable domain of SEQ ID NO: 10 and an antibody heavy chain variable domain of SEQ ID NO: 2;
c) an antibody light chain variable domain of SEQ ID NO: 11 and an antibody heavy chain variable domain of SEQ ID NO: 3;
d) an antibody light chain variable domain of SEQ ID NO: 12 and an antibody heavy chain variable domain of SEQ ID NO: 4;
e) an antibody light chain variable domain of SEQ ID NO: 13 and an antibody heavy chain variable domain of SEQ ID NO: 5;
f) an antibody light chain variable domain of SEQ ID NO: 14 and an antibody heavy chain variable domain of SEQ ID NO: 6;
g) an antibody light chain variable domain of SEQ ID NO: 15 and an antibody heavy chain variable domain of SEQ ID NO: 7; or
h) an antibody light chain variable domain of SEQ ID NO: 16 and an antibody heavy chain variable domain of SEQ ID NO: 8;

6. An antibody that is able to block binding of the binding compound of claim 5 to human REG4 in a cross-blocking assay.

7. An isolated nucleic acid encoding at least one of the light chain variable domain or heavy chain variable domain of the binding compound of claim 5.

8. An expression vector comprising the nucleic acid of claim 7 operably linked to control sequences that are recognized by a host cell when the host cell is transfected with the vector.

9. A host cell comprising the expression vector of claim 8.

10. A method of producing a polypeptide comprising:
culturing the host cell of claim 9 in culture medium under conditions wherein the nucleic acid sequence is expressed, thereby producing polypeptides comprising the light and heavy chain variable domains; and
recovering the polypeptides from the host cell or culture medium.

11. The binding compound of claim 5, further comprising:
a) human germline light chain framework sequences in the antibody light chain variable domain; and
b) human germline heavy chain framework sequences in the antibody heavy chain variable domain.

12. The binding compound of claim 5, further comprising a heavy chain constant region comprising a γ1 human heavy chain constant region or a variant thereof, wherein the constant region variant comprises up to 20 conservatively modified amino acid substitutions.

13. The binding compound of claim 5, further comprising a heavy chain constant region comprising a γ4 human heavy chain constant region or a variant thereof, wherein the constant region variant comprises up to 20 conservatively modified amino acid substitutions.

14. The binding compound of claim 5, wherein the binding compound is an antibody fragment selected from the group consisting of Fab, Fab′, Fab′-SH, Fv, scFv, F(ab′)₂, and a diabody.

15. A method of treating a proliferative disorder in a human subject comprising administering to a subject in need thereof an antibody specific for REG4, or a antigen binding fragment thereof, in an amount effective to inhibit at least one biological activity of REG4, wherein the antibody is the antibody of claim 5.

16. The method of claim 15, wherein the proliferative disorder is a cancer or a tumor.

17. A method of inhibiting angiogenesis in a human subject comprising administering to a subject in need thereof an antibody specific for REG4, or a antigen binding fragment thereof, in an amount effective to inhibit the biological activity REG4, wherein the antibody is the antibody of claim 5.

18. A pharmaceutical composition comprising the antibody of claim 5 and a pharmaceutically acceptable carrier.

19. A method of screening for an inhibitor of REG4 comprising:
a) contacting a cell with a REG4 antagonist;
b) measuring at least one phosphorylation event in the cell; and
c) selecting the REG4 inhibitor that decreases phosphorylation of at least one molecule expressed by the cell.

20. The method of claim 19, wherein the REG4 antagonist is a REG4 antibody or fragment thereof.


SEQUENCE LISTING (65 sequences)

SEQ ID NO: 1 - protein, 141 aa, Rattus norvegicus
MNFGLSLIFLVLVLKGVLCEVKVVASGGILVQPGGSLKLSCIVSGFTFRSYIMSWVRQNPEKRLEWVAEINDGGSYTHYPDTVKGRFIVSRDNDNNILYLQMNSLKSEDTAMYYCVRHGSNGYDYFGMDYWGQGTSVTVSS

SEQ ID NO: 2 - protein, 141 aa, Rattus norvegicus
MNFGLSLIFLVLVLKGVLCEVKVVESGGGLVQPGESLKLSCTASGFTFSTYVMSWIRQTPEKRLEWVAEINDGGAYTHYPDTVEGRFTISRDNDNNTLYLEMSSLKSEDTAMYYCVRHGSDGYNYYGMDHWGQGTSVTVSS

SEQ ID NO: 3 - protein, 141 aa, Rattus norvegicus
MNFGLSLIFLVLVLKGVLCEVKVVASGGILVQPGGSLKLSCTASGFTFRSYVMSWVRQNPEKRLEWVAEINDGGSYTHYPDSVKGRFIISRDNDNHILYLQMNSLKSEDTAMYYCVRHGSTGYDYFGMDYWGQGTSVTVSS

SEQ ID NO: 4 - protein, 134 aa, Rattus norvegicus
MKVLSLLYLLTAIPGILSDVQLLESGPGLVRPSQSLSLTCSVTGNSITSYYWTWIRQFPGNKLEWMGYIGYDDSNEYNPSLKNRISITRDTSKNQFFLKLNSVTSEDTATYFCAREDYDWFAYWGQGTLVTVSA

SEQ ID NO: 5 - protein, 138 aa, Rattus norvegicus
MAWVWTLLFLMAAAQSAQAQIQLVQSGPELKKPGETVKISCKASGYTFTNYGMNWVKQAPGKGLKWMGWINTYTGEPTYAADFKGRFALSLETSASTAYLQINNLKNEDTATYLCARRNWDPYALDYWGQGTSVTVSS

SEQ ID NO: 6 - protein, 141 aa, Rattus norvegicus
MNFGLSLIFLVLVLKGVLCEVKVVESGGGLVQPGGSLKLSCTASGFSFGTYIMSWIRQTPEKRLEWVAEISDGGSYTHYPDTVKGRFIISRDNDKNTLYLQMSSLTSEDTAMYYCARHGSTGYDYFGMDYWGQGTSVTVSS

SEQ ID NO: 7 - protein, 135 aa, Rattus norvegicus
MEWRWIFLFLLSGTTGVHSEIQLQQSGPELVKPGASVKVSCKASGYAFTSYNMYWVKQSHGQSLEWIGRIDPHNGVTTYNQKFKGKPTLTVDRSSSTAFMHLNSLTSEDSAVYYCAREGYDYDVWGQGTTLTVSS

SEQ ID NO: 8 - protein, 135 aa, Rattus norvegicus
MAWVWTLLFLMAAAQSIQAQIQLVQSGPELKKPGETVKISCKASGYTFTDYSMHWVKQAPGKGLKWMGWINTETGEPTYADDFKGRFAFSLETSASTAYLQINNLKNEDTATFFCGRDPYAMDYWGQGTSVTVSS

SEQ ID NO: 9 - protein, 132 aa, Rattus norvegicus
MKLPVRLLVLMFWIPASWSDVLMTQTPLSLSVSLGDQASISCRSSQTIAHRNGITYLEWYLQKPGQSPKLLMYKVSNRFSGVPDRFSGSGSGTDFTLKISRVEAEDLGLYYCFQNSHLPPTFGGGTNLEIKR

SEQ ID NO: 10 - protein, 132 aa, Rattus norvegicus
MKLPVRLLVLMVWIPASRSAVLMTQTPLSLPVSLGDQASISCRSSQNIVHNNGITYLEWFLQKPGQSPKLLMYKVSNRFSGVPDRFSGSGSGTDFTLKISRVEAEDLGVYYCFQNSHLPPTFGGGTILEIKR

SEQ ID NO: 11 - protein, 132 aa, Rattus norvegicus; identical to SEQ ID NO: 9

SEQ ID NO: 12 - protein, 128 aa, Rattus norvegicus
MVFTPQILGLMLFWISASRGDIVLTQSPVILSVTPGDSVSLSCRASQSIGNNLHWYHQKSHESPRLLIKYVSQSISGIPSRFSGSGSETGFTLRLNSVETEDFGMFFCQQSNSWPFTFGSGTKLEIKR

SEQ ID NO: 13 - protein, 134 aa, Rattus norvegicus
MESQTQVLMSLLFWVSGTCGDIVMTQSPSSLTVTAGEKFTMSCKSSQSLVNSGNQKNFLTWYQQKPGQPPKLLIYWASTRESGVPDRFTGSGSGTDFTLTISSVQAEDLAVYYCQNDYSYPYTFGGGTKLEIKR

SEQ ID NO: 14 - protein, 132 aa, Rattus norvegicus
MKLPVRLLVLMFWIPASRSDVLMTQTPLSLPVNLGDQASISCRSSQSIVHNNGITYLEWFLQKPGQSPKLLMYKVSNRFSGVPDRFSGSGSGTEFTLKISRVEAEDLGVYYCFQGSHVPPTFGGGTKLEIKR

SEQ ID NO: 15 - protein, 128 aa, Rattus norvegicus
MRTPAQFLGILLLWFPGIKCDIKMTQSPSSMYASLGERVSITCKASQDINSFLSWFQQKPGKSPKTLIYRANGLVDGVPSRFSGSGSGQDYSLTISSLEYEDMGIYYCLQYDEFPYTFGGGTKLEIKR

SEQ ID NO: 16 - protein, 132 aa, Rattus norvegicus
MKLPVRLLVLMFWIPASSSDVVMTQTPLSLPVSLGDQASISCRSSQSLVHIIGITYLHWYLQKPGQSPKLLIYKVSKRYSGVPDRFSGSGSGTDFTLKISRVEAEDLGVYFCSQSTHVPPTFGAGTKLELKR

SEQ ID NO: 17 - protein, 10 aa, Rattus norvegicus
GFTFRSYIMS

SEQ ID NO: 18 - protein, 10 aa, Rattus norvegicus
GFTFSTYVMS

SEQ ID NO: 19 - protein, 10 aa, Rattus norvegicus
GFTFRSYVMS

SEQ ID NO: 20 - protein, 10 aa, Rattus norvegicus
GNSITSYYWT

SEQ ID NO: 21 - protein, 10 aa, Rattus norvegicus
GYTFTNYGMN

SEQ ID NO: 22 - protein, 10 aa, Rattus norvegicus
GFSFGTYIMS

SEQ ID NO: 23 - protein, 10 aa, Rattus norvegicus
GYAFTSYNMY

SEQ ID NO: 24 - protein, 10 aa, Rattus norvegicus
GYTFTDYSMH

SEQ ID NO: 25 - protein, 17 aa, Rattus norvegicus
EINDGGSYTHYPDTVKG

SEQ ID NO: 26 - protein, 17 aa, Rattus norvegicus
EINDGGAYTHYPDTVEG

SEQ ID NO: 27 - protein, 17 aa, Rattus norvegicus
EINDGGSYTHYPDSVKG

SEQ ID NO: 28 - protein, 16 aa, Rattus norvegicus
YIGYDDSNEYNPSLKN

SEQ ID NO: 29 - protein, 17 aa, Rattus norvegicus
WINTYTGEPTYAADFKG

SEQ ID NO: 30 - protein, 17 aa, Rattus norvegicus
EISDGGSYTHYPDTVKG

SEQ ID NO: 31 - protein, 17 aa, Rattus norvegicus
RIDPHNGVTTYNQKFKG

SEQ ID NO: 32 - protein, 17 aa, Rattus norvegicus
WINTETGEPTYADDFKG

SEQ ID NO: 33 - protein, 17 aa, Rattus norvegicus
EINDGGSYTHYPDTVKG

SEQ ID NO: 34 - protein, 13 aa, Rattus norvegicus
HGSDGYNYYGMDH

SEQ ID NO: 35 - protein, 13 aa, Rattus norvegicus
HGSTGYDYFGMDY

SEQ ID NO: 36 - protein, 8 aa, Rattus norvegicus
EDYDWFAY

SEQ ID NO: 37 - protein, 10 aa, Rattus norvegicus
RNWDPYALDY

SEQ ID NO: 38 - protein, 13 aa, Rattus norvegicus
HGSTGYDYFGMDY

SEQ ID NO: 39 - protein, 7 aa, Rattus norvegicus
EGYDYDV

SEQ ID NO: 40 - protein, 7 aa, Rattus norvegicus
DPYAMDY

SEQ ID NO: 41 - protein, 16 aa, Rattus norvegicus
RSSQTIAHRNGITYLE

SEQ ID NO: 42 - protein, 16 aa, Rattus norvegicus
RSSQNIVHNNGITYLE

SEQ ID NO: 43 - protein, 16 aa, Rattus norvegicus
RSSQTIAHRNGITYLE

SEQ ID NO: 44 - protein, 11 aa, Rattus norvegicus
RASQSIGNNLH

SEQ ID NO: 45 - protein, 17 aa, Rattus norvegicus
KSSQSLVNSGNQKNFLT

SEQ ID NO: 46 - protein, 16 aa, Rattus norvegicus
RSSQSIVHNNGITYLE

SEQ ID NO: 47 - protein, 11 aa, Rattus norvegicus
KASQDINSFLS

SEQ ID NO: 48 - protein, 16 aa, Rattus norvegicus
RSSQSLVHIIGITYLH

SEQ ID NO: 49 - protein, 7 aa, Rattus norvegicus
KVSNRFS

SEQ ID NO: 50 - protein, 7 aa, Rattus norvegicus
KVSNRFS

SEQ ID NO: 51 - protein, 7 aa, Rattus norvegicus
KVSNRFS

SEQ ID NO: 52 - protein, 7 aa, Rattus norvegicus
YVSQSIS

SEQ ID NO: 53 - protein, 7 aa, Rattus norvegicus
WASTRES

SEQ ID NO: 54 - protein, 7 aa, Rattus norvegicus
KVSNRFS

SEQ ID NO: 55 - protein, 7 aa, Rattus norvegicus
RANGLVD

SEQ ID NO: 56 - protein, 7 aa, Rattus norvegicus
KVSKRYS

SEQ ID NO: 57 - protein, 9 aa, Rattus norvegicus
FQNSHLPPT

SEQ ID NO: 58 - protein, 9 aa, Rattus norvegicus
FQNSHLPPT

SEQ ID NO: 59 - protein, 9 aa, Rattus norvegicus
FQNSHLPPT

SEQ ID NO: 60 - protein, 9 aa, Rattus norvegicus
QQSNSWPFT

SEQ ID NO: 61 - protein, 9 aa, Rattus norvegicus
QNDYSYPYT

SEQ ID NO: 62 - protein, 9 aa, Rattus norvegicus
FQGSHVPPT

SEQ ID NO: 63 - protein, 9 aa, Rattus norvegicus
LQYDEFPYT

SEQ ID NO: 64 - protein, 9 aa, Rattus norvegicus
SQSTHVPPT

SEQ ID NO: 65 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP